(19) United States
(12) Patent Application Publication
(10) Pub. No.: US 2026/0272832 A1
(43) Pub. Date: Sep. 17, 2026

(54) A BIPHASIC LIQUID PHARMACEUTICAL COMPOSITION OF AN ANTICONVULSANT DRUG AND THE PROCESS OF PREPARING THE SAME

(71) Applicant: ORBIT PHARMA LIMITED, London (GB)

(72) Inventor: Kamleshkumar Patel, London (GB)

(21) Appl. No.: 19/168,852

(22) PCT Filed: Mar. 26, 2024
(86) PCT No.: PCT/GB2024/050808

§ 371 Date: Sep. 25, 2025

(30) Foreign Application Priority Data
Mar. 29, 2023 (GB) 2304640.2

(51) Int. Cl. A61K 9/10; A61K 31/4166; A61K 31/53; A61K 31/55; A61K 47/10; A61K 47/12; A61K 47/14; A61K 47/22; A61K 47/32

(52) U.S. Cl. CPC A61K 9/10; A61K 31/4166; A61K 31/53; A61K 31/55; A61K 47/10; A61K 47/12; A61K 47/14; A61K 47/22; A61K 47/32

(57) ABSTRACT

The present invention relates to biphasic liquid pharmaceutical composition of an Anticonvulsant drug or pharmaceutically acceptable salts thereof. The biphasic liquid pharmaceutical composition comprising Anticonvulsant drug or pharmaceutically acceptable salts thereof, at least one Preservative, a suspending agent, a Dispersing agent, a sweetener, a pH adjusting agent, an Anti-foaming agent, a flavoring agent, and a vehicle. The Biphasic liquid pharmaceutical composition of an Anticonvulsant drug provides improved palatability particularly for pediatrics, geriatric and dysphasic patients, and aids in avoiding swallowing challenges and for the ease of administration of bitter drugs to the patients.

### FIELD OF THE INVENTION

The present invention relates to a liquid pharmaceutical composition of an Anticonvulsant drug. The present invention more particularly relates to a Biphasic liquid pharmaceutical composition of an Anticonvulsant drug for Oral administration. The present invention relates to the process of preparation of an anticonvulsant drug.

### BACKGROUND OF THE INVENTION

Anticonvulsant drugs act on diverse molecular targets to selectively modify the excitability of neurons so that seizure related firing can be blocked without disturbing non-epileptic activity which serves normal signals between neurons. At the cellular levels, Anticonvulsant drugs act on the modulation of voltage-dependent ion channels (Na+, Ca2+, K+), enhancement of GABA-mediated inhibitory neurotransmission, and attenuation of excitatory transmission.

Anticonvulsant drugs are indicated in psychiatric conditions. These include mainly alcohol and benzodiazepine withdrawal syndromes, panic and anxiety disorders, dementia, schizophrenia, epilepsy, bipolar disorder, partial seizures, primary and secondary tonic-clonic seizures, and seizures associated with Lennox-Gastaut syndrome and personality disorders.

Anticonvulsant drugs disclosed in the present invention are poorly soluble in water and possess high permeability which makes them belong to BCS Class-II drugs. Therefore, formulating the anticonvulsant drugs as Biphasic Liquid pharmaceutical compositions can be useful to solubilize low-solubility drugs.

Anticonvulsant drugs disclosed in the present invention are Lamotrigine, phenytoin and Carbamazepine are currently available as tablets, capsules, chewable dispersible tablets, tablets for oral suspension, orally disintegrating tablets, and Extended-release tablets and capsules, suspensions and solutions. The marketed solid dosage forms of Lamotrigine, phenytoin and carbamazepine are available in strengths as follows: 2 mg, 5 mg, 25 mg, 50 mg, 100 mg, 150 mg, 200 mg, 300 mg, and 400 mg. Liquid formulations like suspensions and solutions of Lamotrigine, phenytoin and carbamazepine are available in the market in the following strengths 5 mg/ml, 10 mg/ml, 20 mg/ml, 30 mg/5 ml, 50 mg/ml, 100 mg/5 ml, and 125 mg/5 ml. The conventional dosage forms of the mentioned Anticonvulsant drugs available in the market are not ready-to-use formulations.

The marketed solid dosage forms of an Anticonvulsant drug are recommended for administration in patients thrice per day leading to problems like patient non-compliance and challenges like the bitter taste of the Anticonvulsant drugs. Therefore, anticonvulsant drugs are suitable candidates for formulating as biphasic liquid pharmaceutical compositions to avoid swallowing challenges, to provide flexible and accurate dosing, for taste-masking of bitter drugs, for improved palatability, particularly for pediatrics, geriatric and dysphasic patients and ease of administration of such bitter drugs to patients. Furthermore, Lamotrigine is available in Tablets for Suspension form in market which requires crushing of tablets to powder form. Such extemporaneous preparations are more to prone to result in concerns like inaccurate dosing and contamination which can be fatal for Patients. In order to solve the aforementioned disadvantages, the present invention is aimed for providing a ready-to-use Biphasic liquid pharmaceutical composition of Lamotrigine, phenytoin and carbamazepine.

US20200046716A1 discloses the suspension and suspension powder for reconstitution comprising lamotrigine and the process for its preparation. The manufacturing process involves the addition of sucrose and sorbitol to hot water to form a solution. Thereafter, Lamotrigine and a previously mixed solution of Xanthan gum and Avicel were added. Sodium saccharin, monosodium dibasic phosphate, potassium sorbate and/or sodium benzoate, methyl paraben, propyl paraben, and desired flavor were added to the final solution with stirring and volume was made with water to form a suspension.

US20210069109A1 discloses an oral liquid suspension that includes lamotrigine and methods that include administering the oral liquid suspension. The suspension was formulated using Lamotrigine as the active ingredient and inactive ingredients included water, glycerin, propylene glycol, polyethylene glycol 400, methyl paraben, sodium benzoate, sorbitol, saccharin sodium, sucralose, xanthan gum, sodium carboxymethyl cellulose, sodium phosphate dibasic, MCC, a coloring agent; and a flavoring agent.

U.S. Pat. No. 4,280,995A discloses an oral suspension of phenytoin. The manufacturing process involves admixing solutions of guaran and phenytoin to form a suspension. Phenytoin is triturated with anhydrous ethanol. Then to this phenytoin-alcohol mixture, a solution of sorbitol was added. Thereafter with stirring, guaran solution is added to the final solution forming a chemical compound. To this complex, sufficient distilled water is added to form a final suspension.

U.S. Pat. No. 10,653,626B2 discloses a powder formulation for reconstitution with a pharmaceutically acceptable carrier to form a suspension of lamotrigine. Dihydrate dihydrogen phosphate, polyethylene glycol, sucralose, strawberry essence, potassium sorbate, and maltitol were added successively into purified water with a stirring and then carrageenan was added into a pre-mixed solution with constant stirring. Thus, to the final solution mixture, Lamotrigine was added slowly into the suspension with stirring to form a suspension.

US20210196623A1 discloses a solution of carbamazepine cyclodextrin inclusion complex useful for the parenteral administration of carbamazepine. The manufacturing process involves hydroxyl propyl beta cyclodextrin was dissolved in deionized water to generate a solution. Then, carbamazepine was added to this solution. The resulting solution was stirred for 24 hours at room temperature. The resulting solution was filtered into a sterile receiver.

### SUMMARY OF THE INVENTION

In accordance with the present invention, a Biphasic Liquid pharmaceutical composition for oral administration is prepared. A Biphasic liquid pharmaceutical composition comprises an Anticonvulsant drug, at least one Preservative, at least one suspending agent, a dispersing agent, a sweetener, a pH adjusting agent, an Antifoaming agent and a vehicle. A Biphasic liquid pharmaceutical composition further comprises one or more pharmaceutically acceptable excipients.

Further, another embodiment of the present invention involves a process for preparing a Biphasic liquid pharmaceutical composition comprising an Anticonvulsant drug for oral administration. The Biphasic Liquid pharmaceutical composition is prepared by mixing and high-shear homogenization.

Another embodiment of the present invention which effectively treat psychiatric conditions which includes mainly alcohol and benzodiazepine withdrawal syndromes, panic and anxiety disorders, dementia, schizophrenia, epilepsy, bipolar disorder, partial seizures, primary and secondary tonic-clonic seizures, and seizures associated with Lennox-Gastaut syndrome and personality disorders.

### OBJECTS OF THE INVENTION

The principal object of the present invention is to provide a Biphasic Liquid pharmaceutical composition of an Anticonvulsant drug or its pharmaceutically acceptable salts thereof.

It is object of the present invention is to formulate a Biphasic liquid pharmaceutical composition which is best suited liquid dosage form for improving solubility and dissolution rate of poorly water soluble Anticonvulsant drugs or its pharmaceutically acceptable salts thereof which belongs to BCS Class-II.

It is another objective of the present invention is to provide a Biphasic liquid pharmaceutical composition of an Anticonvulsant drug or its pharmaceutically acceptable salts thereof which is ready to use solution over markedly available solid dosage forms.

It is further another object of the present invention to provide a process for the preparation of a stable Biphasic liquid pharmaceutical composition comprising an Anticonvulsant drug or its pharmaceutically acceptable salts thereof.

Yet another object of the present invention is to provide good patient compliance in the case of pediatric, geriatric even dysphasic patients, flexible & accurate dosing, and improved palatability.

### DETAILED DESCRIPTION OF THE INVENTION

The present invention is understood more readily by reading the following detailed description of the invention and by studying the included examples.

Further, the term “Biphasic Liquid”, used in the present invention means a heterogeneous biphasic dosage form and it is a coarse dispersion in which insoluble solid particles are dispersed in a liquid medium. The dispersed phase is distributed in particulate form throughout the continuous phase.

The term “about”, as and when used in this specification, means±10% of the mentioned value.

As per one embodiment of the present invention, an Anticonvulsant drug is selected from the group consisting of Carbamazepine, Oxcarbazepine, Lamotrigine Phenytoin, Zonisamide, Gabapentin, Valproicacid, Levetiracetam, Pregabalin, Clonazepam, Lacosamide, Rufinamide, Vigabatrin, Phenobarbital, Valnoctamide, Perampael, Pyridoxine or pharmaceutically acceptable salts thereof.

As per one embodiment of the present invention, a Biphasic liquid pharmaceutical composition comprising an Anticonvulsant drug or pharmaceutically acceptable salts thereof is Lamotrigine, phenytoin and Carbamazepine. As per one preferred embodiment of the present invention, an Anticonvulsant drug or pharmaceutically acceptable salts thereof is Lamotrigine.

As per one embodiment of the present invention, an Anticonvulsant drug or pharmaceutically acceptable salts thereof is present in the range from about 0.30% w/v to about 4.0% w/v, preferably in the range from about 0.35% w/v to about 3.5% w/v.

As per one embodiment of the present invention, an Anticonvulsant drug in the Biphasic liquid pharmaceutical composition has a D10 particle size distribution from about 1.0 μm to 10.0 μm, preferably in the range from about 2.5 μm to 9.0 μm. The term D10 is the diameter of the particle at which 10% of a sample's mass is comprised of smaller particles in that sample size. In a further embodiment, an Anticonvulsant drug has a D50 particle size distribution from about 10 μm to 30 μm, preferably in the range from about 15.0 μm to 25 μm. The term D50 is the diameter at which 50% of a sample's mass is comprised of smaller particles in that sample size. The term D50 is defined as the median for a volume distribution of the particles. In a more preferred embodiment, an Anticonvulsant drug has a D90 particle size distribution from about 30 μm to 50 μm, preferably in the range from about 35 μm to 45 μm. The term D90 is the diameter at which 90% of a sample's mass is comprised of smaller particles in that sample size.

As per one embodiment of the present invention, the Preservatives are selected from the group consisting of methyl paraben, propyl paraben, butyl paraben and their salts, sorbic acid, sodium sorbate, potassium sorbate, calcium sorbate, benzoic acid, sodium benzoate, potassium benzoate, calcium benzoate, methyl hydroxyl benzoate, ethyl para hydroxyl benzoate, sodium ethyl para hydroxyl benzoate, sodium metabisulphite, chlorhexidine, diazolidinyl urea, sodium citrate, butylated hydroxyl toluene, butylated hydroxyl anisole, or combinations thereof. In the present invention, the preservatives are present in the range from about 0.010% w/v to about 0.40% w/v, preferably in the range from about 0.015% w/v to about 0.30% w/v. The Parabens are the most commonly used preservatives because of their low toxicity to humans and their effective antimicrobial activity, especially against molds and yeasts.

As per one embodiment of the present invention, the suspending agent is selected from the group consisting of alginates, acacia gum, tragacanth, guar gum, bean gum, carrageenan, xanthan gum, microcrystalline cellulose, powdered cellulose, methylcellulose, sodium carboxymethyl cellulose, HPMC, hydroxyl ethyl cellulose, carboxymethylcellulose, Carbomer or combinations thereof. In the present invention, Carbomer is preferred as suspending agent present in the range from about 0.10% w/v to about 3.5% w/v, preferably in the range from about 0.15% w/v to about 2.0% w/v. Carbomer aids in suspending insoluble ingredients, modifying flow characteristics, provides bio adhesion and provide excellent suspending ability and virtually eliminates the problem of settling, even when used at very low levels.

As per one another embodiment of the present invention, the Dispersing agent is selected from the group consisting of polyvinyl pyrrolidone, sodium hexameta phosphate, the sodium salt of EDTA, Glycerol, sodium dodecyl sulfonate, sodium dodecyl benzene sulfonate, polysorbates, Sorbitan esters, sodium lauryl sulphate, sodium dioctyl sulphosuccinate or combinations thereof. Dispersing agents are added to liquid formulations to stabilize these formulations by preventing the aggregation of the suspended particles. In the present invention, Glycerol is preferred as dispersing agent in the range from about 2.5% w/v to about 7.5% w/v, preferably in the range from about 3.5% w/v to about 6.5% w/v.

As per one more embodiment of the present invention, a suitable sweetener is selected from the group consisting of Acesulfame potassium, Alitame, Aspartame, Dextrose, Fructose, Galactose, Inulin, Isomalt, Lactitol, Maltitol, Maltose, Mannitol, Saccharin, Sodium cyclamate, Sorbitol, Sucralose, Sucrose, Tagatose, Thaumatin, Trehalose, Xylitol or combinations thereof. Sodium saccharin is preferred as a sweetener present in the range from about 0.05% w/v to about 0.090% w/v, preferably in the range from about 0.065% w/v to about 0.085% w/v. Sweeteners are added to pharmaceutical dosage forms to mask the bitter taste of the partially dissolved drug and to improve palatability.

As per one embodiment of the present invention, the Anti-foaming agents are selected from the group consisting of cetostearyl alcohol, insoluble oils, stearates, polydimethylsiloxanes and other silicones derivatives, organic phosphates, paraffin oils, stearate, Simethicone, ethers, glycols or combinations thereof. Anti-foaming agents discourage the formation of stable foam by lowering the surface tension and cohesive binding of the liquid phase. In the present invention, Anti-foaming agents are present in the range from about 0.025% w/v to about 0.060% w/v, preferably in the range from about 0.035% w/v to about 0.065% w/v.

As per one embodiment of the present invention, the flavoring agent is selected from the group consisting of banana, orange, grape, lime and grapefruit, vanilla, and fruit essence, peppermint, lemon oils, butterscotch, apricot; synthetic flavor oils and flavoring aromatics and natural oils, extracts from plant leaves, cinnamon oil, peppermint oils, clove oil, citrus oil, bay oil, oil of bitter almonds, maltol, ethyl vanillin, menthol, citric acid, fumaric acid, ethyl maltol and tartaric acid or combinations thereof. Flavoring agents are generally provided as a minor component of the suspension in amounts effective to provide a palatable flavor to the suspension. In the present invention, Natural Strawberry flavor is preferred as flavoring agent in the range from about 0.05% w/v to about 0.20% w/v, preferably in the range from about 0.070% w/v to about 0.15% w/v.

As per one embodiment of the present invention, the pH adjusting agents are selected from the group consisting of Citrates, phosphates, hydrochloric acid, Trolamine, malic acid, Potassium metaphosphate, sodium hydroxide, tartaric acid, Sodium lactate, Potassium citrate, sulfuric acid, sodium glycolate, Boric acid, Potassium chloride, Citric acid, Sodium Citrate, gluconates, succinate, acetate or combinations thereof. The pH adjusting agents are added to suspensions to control potential changes in the pH of formulations. In the preferred embodiments of the present invention, the pH of the Biphasic Liquid pharmaceutical composition is from 3.0 to 8.0, preferably from 4.5 to 7.5.

As per one more embodiment of the present invention, the vehicle is selected from the group consisting of purified water, alcohol, polyethylene glycol, propylene glycol or glycerin buffers, oils, acetylated glycerides, ethyl oleate, mineral oil, fatty acid esters, mono- or di-fatty acid esters of polyethylene glycols, or glyceryl Monooleate, alcohol, acetic acid, acetone, ethyl acetates or combinations thereof. vehicles are used as a base in which drugs and other excipients are dissolved or dispersed. Purified Water is used commonly in Liquid formulations due to its lack of toxicity, physiological compatibility and good solubilizing power. Purified water is preferred as the vehicle in the present invention.

As per preferred embodiment of the present invention, a Biphasic liquid pharmaceutical composition comprising an Anticonvulsant drug or pharmaceutically acceptable salts thereof is present in the range from about 0.30% w/v to about 4.0% w/v, preferably in the range from about 0.35% w/v to about 3.5% w/v, the preservatives present in the range from about 0.010% w/v to about 0.40% w/v, preferably in the range from about 0.015% w/v to about 0.30% w/v, a suspending agent is present in the range from about 0.10% w/v to about 3.5% w/v, preferably in the range from about 0.15% w/v to about 2.0% w/v, a dispersing agent present in the range from about 2.5% w/v to about 7.5% w/v, preferably in the range from about 3.5% w/v to about 6.5% w/v, the pH of said composition is from 3.0 to 8.0, preferably from 4.5 to 7.5 and vehicles or combinations thereof.

As another preferred embodiment of the present invention, a Biphasic liquid pharmaceutical composition comprising an Anticonvulsant drug or pharmaceutically acceptable salts thereof is present in the range from about 0.30% w/v to about 4.0% w/v, preferably in the range from about 0.35% w/v to about 3.5% w/v, the preservatives are present in the range from about 0.010% w/v to about 0.40% w/v, preferably in the range from about 0.015% w/v to about 0.30% w/v, a suspending agent is present in the range from about 0.10% w/v to about 3.5% w/v, preferably in the range from about 0.15% w/v to about 2.0% w/v, a dispersing agent present in the range from about 2.5% w/v to about 7.5% w/v, preferably in the range from about 3.5% w/v to about 6.5% w/v, the pH of said composition is from 3.0 to 8.0, preferably from 4.5 to 7.5 and vehicles or combinations thereof. The Biphasic liquid pharmaceutical composition further comprises at least one pharmaceutically acceptable excipient selected from the sweetener present in the range from about 0.05% w/v to about 0.090% w/v, preferably in the range from about 0.065% w/v to about 0.085% w/v, Anti-foaming agents are present in the range from about 0.025% w/v to about 0.060% w/v, preferably in the range from about 0.035% w/v to about 0.065% w/v, flavoring agent is present in the range from about 0.05% w/v to about 0.20% w/v, preferably in the range from about 0.07% w/v to about 0.15% w/v.

As per one embodiment of the present invention, the ratio of an Anticonvulsant drug to suspending agent is in the range from about 0.5:1 to about 15:1 and preferably it is in the range from about 1.5:1 to about 10:1.

As per one embodiment of the present invention, the process for the preparation of Biphasic Liquid pharmaceutical composition containing an Anticonvulsant drug or pharmaceutically acceptable salts thereof, by mixing and high-shear homogenization. This method is one of the most economical method.

As per one embodiment of the present invention, more than 90% of an Anticonvulsant drug from said composition is released in 15 minutes, preferably more than 90% of an Anticonvulsant drug from said composition is released within 10 minutes.

As per one embodiment of the present invention, a Biphasic Liquid pharmaceutical composition of an Anticonvulsant drug which will be effective in the treatment of psychiatric conditions. These include mainly alcohol and benzodiazepine withdrawal syndromes, panic and anxiety disorders, dementia, schizophrenia, epilepsy, bipolar disorder, partial seizures, primary and secondary tonic-clonic seizures, and seizures associated with Lennox-Gastaut syndrome and personality disorders.

As per one embodiment of the present invention, a Biphasic Liquid pharmaceutical composition of Anticonvulsant drugs or its pharmaceutically acceptable salts thereof is manufactured by mixing and high-shear homogenization. Methyl para hydroxy benzoate and Propyl para hydroxy benzoate were added to previously heated 20% of purified water at 90° C. and dissolved to get clear solution. To this solution, 30% of total volume of purified water was added and the solution was allowed to cool down to room temperature. To this solution, Simethicone emulsion 30% was added and mixed by silverson mixer for 10-15 minutes to get hazy solution. To this solution, Carbomer was added with continuous stirring till it gets uniformly dispersed in the solution. Lamotrigine was separately added and disperse into glycerol and mixed by silverson mixer for 10-15 minutes to get homogenous solution. To the Carbomer containing solution, homogeneous solution of Lamotrigine was added and mixed by silverson mixer with nitrogen purging for 15-20 minutes to get an off-white to white colour homogeneous solution. To this solution, Saccharin sodium was added and mixed by silverson mixer with nitrogen purging for 10-15 minutes to get an off-white to white color homogeneous solution. The pH of solution was measured and adjusted to pH between 6.7-7.0 with Sodium hydroxide solution (IN) with mixing by silverson mixer with nitrogen purging for 15-20 minutes to get an off-white to white color homogeneous solution. To this solution, Natural Strawberry flavor was added and mixed by silverson mixer with nitrogen purging for 15-20 minutes to get an off-white to white color homogeneous solution. The volume of resulting solution was made up with purified water. Final solution was filled in 150 ml amber colored bottle.

The invention is further illustrated by the following examples, which are by no means intended to limit the scope of the invention but are given by way of illustration.

### Example 1

A Biphasic liquid pharmaceutical composition of an Anticonvulsant drug was prepared according to the general formula as shown in Table-I:

TABLE-I

| Sr. No | Name of Ingredient(s) | Quantity (% w/v) |
|---|---|---|
| 1. | Anticonvulsant Drug | 0.35-3.5 |
| 2. | Preservatives | 0.015-0.30 |
| 3. | Suspending agents | 0.15-2.0 |
| 4. | Dispersing agents | 3.5-6.5 |
| 5. | Sweetener | 0.065-0.085 |
| 6. | Anti-foaming agents | 0.035-0.065 |
| 7. | Flavoring agents | 0.070 -0.15 |
| 8. | pH adjusting agents | QS to adjust pH 4.5-7.5 |
| 9. | Vehicles | Upto 100 ml |

### Manufacturing Process:

Preservatives were added to previously heated 20% of purified water at 90° C. and dissolved to get clear solution. To this solution, 30% of total volume of purified water was added and the solution was allowed to cool down to room temperature. To this Solution, Anti-foaming agent was added and mixed by silverson mixer for 10-15 minutes to get hazy solution. To this solution, suspending agent was added with continuous stirring till it gets uniformly dispersed in the solution. Anticonvulsant drug was separately mixed with dispersing agent by silverson mixer for 10-15 minutes to get homogenous solution. To the solution containing suspending agent, homogeneous Solution of Anticonvulsant drug was added and mixed by silverson mixer with nitrogen purging for 15-20 minutes to get an off-white to white color homogeneous Solution. To this resulting Solution, sweetener was added and mixed by silverson mixer with nitrogen purging for 10-15 minutes to get an off-white to white color homogeneous Solution. The pH of solution was measured and adjusted to pH between 6.7-7.0 with Sodium hydroxide solution (IN) with mixing by silverson mixer with nitrogen purging for 15-20 minutes to get an off-white to white color homogeneous solution. To this Solution, flavoring agent was added and mixed by silverson mixer with nitrogen purging for 15-20 minutes to get an off-white to white color homogeneous solution. The volume of final solution was made up with vehicle. The final solution was filled in 150 ml amber colored bottle.

The final solution was measured for pH and taste. The solution had desired pH and acceptable sweet taste.

### Example 2

The manufacturing of Biphasic Liquid pharmaceutical composition of Lamotrigine was conducted according to the method defined in the general formula having the ingredients shown in table-II for different dose strengths that are 5 mg/ml, 25 mg/5 ml, 100 mg/5 ml of Lamotrigine:

TABLE-II

| Name of ingredients | Quantity (% w/v) |
|---|---|
| Lamotrigine | 2.000 |
| Glycerol | 5.000 |
| Carbomer | 0.250 |
| Purified water | To 100 ml |

### Manufacturing Process:

Carbomer was dispersed in the purified water with continuous stirring, till it gets uniformly dispersed in the solution. Lamotrigine was separately added and dispersed into glycerol and mixed by using silverson mixer for 10-15 minutes to get homogenous Solution. To the Carbomer containing solution, homogeneous Solution of Lamotrigine was added and mixed by using silverson mixer with nitrogen purging for 15-20 minutes to get an off-white to white color homogeneous Solution. The volume of final composition was made up with purified water.

The pH of the final solution was 5.8 and required pH was 6.7-7.0. Therefore, the next formulation was prepared with the addition of the pH adjusting agent such as sodium hydroxide (IN solution).

### Example 3

The manufacturing of Biphasic Liquid pharmaceutical composition of Lamotrigine was conducted according to the method defined in the general formula having the ingredients shown in table-III for different dose strengths that are 5 mg/ml, 25 mg/5 ml, 100 mg/5 ml of Lamotrigine:

TABLE-III

| Name of ingredients | Quantity (% w/v) |
|---|---|
| Lamotrigine | 2.000 |
| Glycerol | 5.000 |
| Carbomer | 0.250 |
| Sodium hydroxide solution (1N) | QS to pH 6.7-7.0 |
| Purified water | To 100 ml |

### Manufacturing Process:

Carbomer was dispersed in the 70% of total volume of water with continuous stirring till it gets uniformly dispersed in the water. Lamotrigine was separately added and dispersed into glycerol and mixed by silverson mixer for 10-15 minutes to get homogenous Solution. To the Carbomer containing solution, homogeneous Solution of Lamotrigine was added and mixed by silverson mixer with nitrogen purging for 15-20 minutes to get an off-white to white color homogeneous Solution. The pH of Solution was measured and adjusted to pH between 6.7-7.0 with Sodium hydroxide solution (IN) with mixing by silverson mixer with nitrogen purging for 15-20 minutes to get an off-white to white colour homogeneous suspension. The volume of final composition was made up with purified water.

The final solution was measured for pH and Taste. The pH of solution was 6.8. Lamotrigine has a bitter taste, the taste of said composition was awful and not acceptable. Therefore, the next trial was performed by including saccharin sodium 0.07% to mask the taste as well as add flavor.

### Example 4

The manufacturing of Biphasic Liquid pharmaceutical composition of Lamotrigine was conducted according to the method defined in the general formula having the ingredients shown in table-IV for different dose strengths that are 5 mg/ml, 25 mg/5 ml, 100 mg/5 ml of Lamotrigine:

TABLE-IV

| Name of ingredients | Quantity (% w/v) |
|---|---|
| Lamotrigine | 2.000 |
| Carbomer | 0.250 |
| Glycerol | 5.000 |
| Sodium saccharin | 0.070 |
| Strawberry flavour | 0.100 |
| Sodium hydroxide solution (1N) | QS to pH 6.7-7.0 |
| Purified water | To 100 ml |

### Manufacturing Process:

Carbomer was dispersed in the 70% of total volume of water with continuous stirring till it gets uniformly dispersed in the water. Lamotrigine was separately added and dispersed into glycerol and mixed by silverson mixer for 10-15 minutes to get homogenous Solution. To the Carbomer containing solution, homogeneous Solution of Lamotrigine was added and mixed by using silverson mixer with nitrogen purging for 15-20 minutes to get an off-white to white color homogeneous Solution. To this resulting Solution, Saccharin sodium was added and mixed by silverson mixer with nitrogen purging for 10-15 minutes to get an off-white to white color homogeneous Solution. The pH of final Solution was measured and adjusted to pH between 6.7-7.0 with Sodium hydroxide solution (IN) with mixing by silverson mixer with nitrogen purging for 15-20 minutes to get an off-white to white color homogeneous solution. To this Solution, Natural Strawberry flavor was added and mixed by silverson mixer with nitrogen purging for 15-20 minutes to get an off-white to white color homogeneous said composition. The volume of final composition was made up with purified water.

The final solution was measured for pH and taste. The pH of final solution was 6.8 and it had an acceptable sweet taste. But during mixing foam was generated, Therefore the next trial was performed by including anti foaming agent Simethicone 0.05% to minimise generated foam.

### Example 5

The manufacturing of Biphasic Liquid pharmaceutical composition of Lamotrigine was conducted according to the method defined in the general formula having the ingredients shown in Table-V for different dose strengths that are 5 mg/ml, 25 mg/5 ml, 100 mg/5 ml of Lamotrigine:

TABLE-V

| Name of ingredients | Quantity (% w/v) |
|---|---|
| Lamotrigine | 2.000 |
| Carbomer | 0.250 |
| Glycerol | 5.000 |
| Simethicone Emulsion 30% | 0.050 |
| Sodium saccharin | 0.070 |
| Strawberry flavour | 0.100 |
| Sodium hydroxide solution (1N) | QS to pH 6.7-7.0 |
| Purified water | To 100 ml |

### Manufacturing Process:

Carbomer was dispersed in the 70% of total volume of water with continuous stirring till it gets uniformly dispersed in the water. To this Solution, Simethicone emulsion 30% was added and mixed by silverson mixer for 10-15 minutes to get hazy solution. To the Carbomer containing solution, homogeneous Solution of Lamotrigine was added and mixed by silverson mixer with nitrogen purging for 15-20 minutes to get an off-white to white color homogeneous solution. To this Solution, saccharin sodium was added and mixed by silverson mixer with nitrogen purging for 10-15 minutes to get an off-white to white color homogeneous Solution. The pH of Solution was measured and adjusted to pH between 6.7-7.0 with Sodium hydroxide solution (IN) with mixing by silverson mixer with nitrogen purging for 15-20 minutes to get an off-white to white color homogeneous solution. To this Solution, Natural Strawberry flavor was added and mixed by silverson mixer with nitrogen purging for 15-20 minutes to get an off-white to white color homogeneous solution. The volume of final composition was made up with purified water.

The final solution was measured for pH and taste. The pH of final solution was 6.8 and it had an acceptable sweet taste as well as flavor. But microbial growth was observed in the formulation. Therefore, the next trial was carried out by including the preservatives methyl hydroxyl benzoate and propyl hydroxyl benzoate.

### Example 6

The manufacturing of Biphasic Liquid pharmaceutical composition of Lamotrigine was conducted according to the method defined in the general formula having the ingredients shown in table-VIA and table-VIB for different dose strengths that are 5 mg/ml, 25 mg/5 ml, 100 mg/5 ml of Lamotrigine:

TABLE-VIA

| | | Lamotrigine |
|---|---|---|
| | Name | 25 mg/5 ml oral |
| Sr. | of | suspension |
| No | Ingredient(s) | % W/V |
| 1 | Lamotrigine | 0.500 |
| 2 | Methyl Parahydroxybenzoate | 0.180 |
| 3 | Propyl Parahydroxybenzoate | 0.020 |
| 4 | Carbomer | 0.250 |
| 5 | Glycerol | 5.000 |
| 6 | Sodium saccharin | 0.070 |
| 7 | Simethicone emulsion 30% | 0.050 |
| 8 | Strawberry flavour | 0.100 |
| 9 | Sodium hydroxide Solution (1N) | Q.S. to 6.7-7.0 |
| 10 | Purified water | Up to 100ml |

TABLE-VIB

| | | Lamotrigine |
|---|---|---|
| | | 100 mg/5 ml oral |
| | | Biphasic liquid |
| | Name | pharmaceutical |
| Sr. | of | composition |
| No | Ingredient(s) | %W/V |
| 1 | Lamotrigine | 2.000 |
| 2 | Methyl Parahydroxybenzoate | 0.180 |
| 3 | Propyl Parahydroxybenzoate | 0.020 |
| 4 | Carbomer | 0.250 |
| 5 | Glycerol | 5.000 |
| 6 | Sodium saccharin | 0.070 |
| 7 | Simethicone emulsion 30% | 0.050 |
| 8 | Strawberry flavour | 0.100 |
| 9 | Sodium hydroxide Solution (1N) | Q.S. to 6.7-7.0 |
| 10 | Purified water | Up to 100ml |

### Manufacturing Process:

Methyl para hydroxyl benzoate and Propyl para hydroxyl benzoate were added to previously heated 20% of purified water at 90° C. and dissolved to get clear solution. To this solution, 30% of total volume of purified water was added and the solution was allowed to cool down to room temperature. To this Solution, Simethicone emulsion 30% was added and mixed by silverson mixer for 10-15 minutes to get hazy solution. To this solution, Carbomer was added with continuous stirring till it gets uniformly dispersed in the solution. Lamotrigine was separately dispersed into glycerol and then mixed by silverson mixer for 10-15 minutes to get homogenous Solution. To the Carbomer containing solution, homogeneous Solution of Lamotrigine was added and mixed by silverson mixer with nitrogen purging for 15-20 minutes to get an off-white to white color homogeneous Solution. To this Solution, Saccharin sodium was added and mixed by silverson mixer with nitrogen purging for 10-15 minutes to get an off-white to white color homogeneous Solution. The pH of final Solution was measured and adjusted to pH between 6.7-7.0 with Sodium hydroxide solution (IN) with mixing by silverson mixer with nitrogen purging for 15-20 minutes to get an off-white to white color homogeneous solution. To this Solution, Natural Strawberry flavor was added and mixed by silverson mixer with nitrogen purging for 15-20 minutes to get an off-white to white color homogeneous solution. The volume of final composition was made up with purified water. The final Solution obtained was filled in 150 ml Ambered colored bottle.

The final solution was measured for pH and taste. The solution had desired pH and acceptable sweet taste.

### Example 7

The Biphasic Liquid pharmaceutical composition of Phenytoin was manufactured according to the method defined in the general formula having the ingredients shown in table-VII for different dose strengths that 30 mg/5 ml, 125 mg/5 ml of phenytoin:

TABLE-VII

| | | Phenytoin 125 mg/5 ml |
|---|---|---|
| | | Biphasic Liquid |
| | Name | pharmaceutical |
| Sr. | of | composition |
| No | Ingredient(s) | % w/v |
| 1. | Phenytoin | 2.5 |
| 2. | Methyl Para hydroxybenzoate | 0.015-0.30 |
| 3 | Propyl Para hydroxybenzoate | |
| 4. | Carbomer | 0.15-2.0 |
| 5. | Glycerol | 3.5-6.5 |
| 6. | Sodium saccharin | 0.065-0.085 |
| 7. | Silicon Medical | 0.035-0.065 |
| | Antifoam emulsion | |
| 8. | Strawberry flavour | 0.070-0.15 |
| 9. | 10% Citric acid or | Q.S. to pH 3.0 to 5.0 |
| | Sodium Citrate | |
| | Solution | |
| 10. | Purified water | Up to 100 ml |

### Manufacturing Process:

Methyl parahydroxy benzoate and Propyl parahydroxy benzoate were added to previously heated 20% of purified water at 90° C. and dissolved to get clear solution. To this solution, 30% of total volume of purified water was added and the solution was allowed to cool down to room temperature. To this Solution, Silicon Medical Antifoam emulsion was added and mixed by silverson mixer for 10-15 minutes to get hazy solution. To this solution, Carbomer was added with continuous stirring till it gets uniformly dispersed in the solution. Phenytoin was separately added and disperse into glycerol and mixed by silverson mixer for 10-15 minutes to get homogenous Solution. To the Carbomer containing solution, homogeneous Solution of Phenytoin was added and mixed by silverson mixer with nitrogen purging for 15-20 minutes to get an off-white to white color homogeneous Solution. To this Solution, Saccharin sodium was added and mixed by silverson mixer with nitrogen purging for 10-15 minutes to get an off-white to white color homogeneous Solution. The pH of Solution was measured and adjusted to pH between 3.0 to 5.0 with 10% Citric acid or Sodium Citrate Solution with mixing by silverson mixer with nitrogen purging for 15-20 minutes to get an off-white to white color homogeneous solution. To this Solution, Natural Strawberry flavor was added and mixed by silverson mixer with nitrogen purging for 15-20 minutes to get an off-white to white color homogeneous solution. The volume of final composition was made up with purified water. The final Solution obtained was filled in 150 ml amber colored bottle.

The final solution was measured for pH and taste. The pH of the solution was 4.5 and it had an acceptable sweet taste.

### Example 8

The Biphasic Liquid pharmaceutical composition of Carbamazepine was manufactured according to the method defined in the general formula having the ingredients shown in table-VIII for different dose strengths that is 100 mg/5 ml of Carbamazepine:

TABLE-VIII

| | | Carbamazepine - |
|---|---|---|
| | | 100 mg/5 ml |
| | | Biphasic Liquid |
| | Name | pharmaceutical |
| Sr. | of | composition |
| No | Ingredient(s) | % w/v |
| 1. | Carbamazepine | 2.000 |
| 2. | Methyl Parahydroxybenzoate | 0.015-0.30 |
| 3 | Propyl Parahydroxybenzoate | |
| 4. | Carbomer | 0.15-2.0 |
| 5. | Glycerol | 3.5-6.5 |
| 6. | Sodium saccharin | 0.065-0.085 |
| 7. | Silicon Medical | 0.035-0.065 |
| | Antifoam emulsion | |
| 8. | Natural Strawberry flavour | 0.070-0.15 |
| 9. | 10% Citric acid or Sodium | Q.S. to pH 3.0 to 5.0 |
| | Citrate Solution | |
| 10. | Purified water | Up to 100ml |

### Manufacturing Process:

Methyl para hydroxybenzoate and Propyl para hydroxybenzoate were added to previously heated 20% of purified water at 90° C. and dissolved to get clear solution. To this solution, 30% of total volume of purified water was added and the solution was allowed to cool down to room temperature. To this Solution, Silicon Medical Antifoam emulsion was added and mixed by silverson mixer for 10-15 minutes to get hazy solution. To this solution, Carbomer was added with continuous stirring till it gets uniformly dispersed in the solution. Carbamazepine was separately added and disperse into glycerol and mixed by silverson mixer for 10-15 minutes to get homogenous Solution. To the previously mixed Carbomer containing solution, homogeneous Solution of Carbamazepine was added and mixed by silverson mixer with nitrogen purging for 15-20 minutes to get an off-white to white color homogeneous Solution. To this Solution, Saccharin sodium was added and mixed by silverson mixer with nitrogen purging for 10-15 minutes to get an off-white to white color homogeneous Solution. The pH of Solution was measured and adjusted to pH between 3.0 to 5.0 with 10% Citric acid or Sodium Citrate Solution with mixing by silverson mixer with nitrogen purging for 15-20 minutes to get an off-white to white color homogeneous solution. To this Solution, Natural Strawberry flavor was added and mixed by silverson mixer with nitrogen purging for 15-20 minutes to get an off-white to white color homogeneous solution. The volume of final composition was made up with purified water. The final Solution obtained was filled in 150 ml amber colored bottle.

The final solution was measured for pH and taste. The pH of the solution was 4.7 and it had an acceptable sweet taste.

### Example 9

The Dissolution profile of the Biphasic Liquid pharmaceutical composition of an Anticonvulsant drug was prepared according to Example 6. The conditions of dissolution system are as following:
Product Name: Lamotrigine Biphasic Liquid Pharmaceutical Composition
Strengths: 25 mg/5 ml and 100 mg/5 ml
Media: 0.1 N HCL
Apparatus: Apparatus II (Paddle)
Rate of rotation: 50 RPM
Volume: 900 ml
Temperature: 37° C.
Detection: High performance liquid chromatography equipped with UV/PDA Detector at 270 nm

### Example 10

The Biphasic liquid pharmaceutical composition of Lamotrigine was tested for its dissolution profile measured in 900 mL of 0.1 N HCL at 50 RPM in USP II (Paddle) apparatus and subjected to a stability study of 25° C./60% RH and 40° C./75% RH for 3 months. Results are tabulated below:

| Lamotrigine 25 mg/5 ml Biphasic | | | | |
|---|---|---|---|---|
| Liquid Pharmaceutical Composition | | | | |
| | | | 3 month | 3 month |
| | | | 40° C./ | 25° C./ |
| | | Exp. 6 | 75% | 60% |
| Test | Specification | (Initial) | RH | RHI |
| pH | 6.3 to 7.3 | 6.8 | 6.8 | 6.9 |
| Assay | 95.0%-105.0% | 97.1 | 98.0 | 98.5 |
| | of the | | | |
| | labelled amount | | | |
| | of Lamotrigine | | | |
| | 90.0%-110.0% | 103.2 | 105.9 | 107.4 |
| | of the | | | |
| | labelled amount | | | |
| | of Methyl | | | |
| | Para hydroxy | | | |
| | benzoate | | | |
| | 90.0%-110.0% | 99.5 | 100.0 | 100.9 |
| | of the labelled | | | |
| | amount | | | |
| | of Propyl | | | |
| | Para hydroxy | | | |
| | benzoate | | | |
| Dissolution | NLT 75% (Q) | 98.0 | 90.2 | 98.5 |
| | labelled | | | |
| | amount of | | | |
| | Lamotrigine | | | |
| | should | | | |
| | be dissolve | | | |
| | in 45 minutes. | | | |

Lamotrigine 100 mg/5 ml Biphasic liquid Pharmaceutical Composition

| | | | 3 month | 3 month |
|---|---|---|---|---|
| | | Exp. 6 | 40º C./75% | 25° C./60% |
| Test | Specification | (Initial) | RH | RH |
| pH | 6.3 to 7.3 | 6.8 | 6.9 | 6.8 |
| Assay | 95.0%-105.0% of the | 103.5 | 100.4 | 99.0 |
| | labelled amount of Lamotrigine | | | |
| | 90.0%-110.0% of the | 96.9 | 108.4 | 106.4 |
| | labelled amount of Methyl Parahydroxybenzoate | | | |
| | 90.0%-110.0% of thelabelled amount of Propyl | 92.3 | 94.8 | 99.1 |
| | Parahydroxybenzoate | | | |
| Dissolution | NLT 75% (Q) labelled amount of Lamotrigine | 100.4 | 101.0 | 101.4 |
| | should be dissolve in 45 minutes. | | | |

1. A Biphasic Liquid Pharmaceutical Composition of an Anticonvulsant drug for oral administration comprising:
a) Anticonvulsant or pharmaceutically acceptable salts thereof, present in the range from about 0.30% w/v to about 4.0% w/v, preferably in the range from about 0.35% w/v to about 3.5% w/v;
b) at least one preservative;
c) at least one suspending agent present in the range from about 0.10% w/v to about 3.5% w/v, preferably in the range from about 0.15% w/v to about 2.0% w/v;
d) at least one Dispersing agent present in the range from about 2.5% w/v to about 7.5% w/v, preferably in the range from about 3.5% w/v to about 6.5% w/v;
e) at least pH adjusting agent; and
f) at least a vehicle.

2. The Biphasic liquid pharmaceutical composition according to claim 1, wherein the preservatives are selected from the group consisting of methyl paraben, propyl paraben, butyl paraben and their salts, sorbic acid, sodium sorbate, potassium sorbate, calcium sorbate, benzoic acid, sodium benzoate, potassium benzoate, calcium benzoate, methyl hydroxyl benzoate, ethyl para hydroxy benzoate, sodium ethyl para hydroxyl benzoate, sodium metabisulphite, chlorhexidine, diazolidinyl urea, sodium citrate, butylated hydroxyl toluene, butylated hydroxyl anisole, or combinations thereof.

3. The Biphasic liquid pharmaceutical composition according to claim 2, wherein the preservatives are present in the range from about 0.010% w/v to about 0.40% w/v, preferably in the range from about 0.015% w/v to about 0.30% w/v.

4. The Biphasic liquid pharmaceutical composition according to claim 1, wherein the suspending agent is selected from the group consisting of alginates, acacia gum, tragacanth, guar gum, bean gum, carrageenan, xanthan gum, microcrystalline cellulose, powdered cellulose, methylcellulose, sodium carboxymethyl cellulose, HPMC, hydroxyethyl cellulose, carboxymethylcellulose, carbomers or combinations thereof.

5. The Biphasic liquid pharmaceutical composition according to claim 4, wherein the suspending agent is Carbomer.

6. The Biphasic liquid pharmaceutical composition according to claim 1, wherein the dispersing agent is selected from the group consisting of polyvinyl pyrrolidone, sodium hexametaphosphate, the sodium salt of EDTA, Glycerol, sodium dodecyl sulfonate, sodium dodecyl benzene sulfonate, polysorbates, sorbitan esters, sodium lauryl sulphate, sodium dioctyl sulphosuccinate or combinations thereof.

7. The Biphasic liquid pharmaceutical composition according to claim 6, wherein the dispersing agent is Glycerol.

8. The Biphasic liquid pharmaceutical composition according to claim 1, wherein the pharmaceutical composition further comprises at least one or more pharmaceutically acceptable excipients are selected from the group consisting of sweeteners, Anti-foaming agents, and flavoring agents.

9. The Biphasic liquid pharmaceutical composition according to claim 8, wherein the sweetener is selected from the group consisting of Acesulfame potassium, Alitame, Aspartame, Dextrose, Fructose, Galactose, Inulin, Isomalt, Lactitol, Maltitol, Maltose, Mannitol, Saccharin, Sodium cyclamate, Sorbitol, Sucralose, Sucrose, Tagatose, Thaumatin, Trehalose, Xylitol or combinations thereof is present in the range from about 0.05% w/v to about 0.090% w/v, preferably in the range from about 0.065% w/v to about 0.085% w/v.

10. The Biphasic liquid pharmaceutical composition according to claim 8, wherein the Anti-foaming agents are selected from the group consisting of cetostearyl alcohol, insoluble oils, stearates, polydimethylsiloxanes, and other silicones derivatives, organic phosphates, Paraffin oils, Stearates, Simethicone, ethers, glycols or combinations thereof are present in the range from about 0.025% w/v to about 0.060% w/v, preferably in the range from about 0.035% w/v to about 0.065% w/v.

11. The Biphasic liquid pharmaceutical composition according to claim 8, wherein the flavoring agent is selected from the group consisting of banana, orange, grape, lime and grapefruit, vanilla, and fruit essence, peppermint, lemon oils, butterscotch, apricot, synthetic flavor oils and flavoring aromatics, natural oils, extracts from plant leaves, cinnamon oil, peppermint oils, clove oil, citrus oil, bay oil, oil of bitter almonds, maltol, ethyl vanillin, menthol, citric acid, fumaric acid, ethyl maltol, and tartaric acid or combinations thereof is present in the range from about 0.05% w/v to about 0.20% w/v, preferably in the range from about 0.070% w/v to about 0.15% w/v.

12. The Biphasic liquid pharmaceutical composition according to claim 1, wherein the pH adjusting agent is selected from the group consisting of Citrates, phosphates, hydrochloric acid, Trolamine, malic acid, Potassium metaphosphate, sodium hydroxide, tartaric acid, Sodium lactate, Potassium citrate, sulfuric acid, sodium glycolate, Boric acid, Potassium chloride, Citric acid, Sodium Citrate, gluconates, succinate, acetate or combinations thereof.

13. The Biphasic liquid pharmaceutical composition according to claim 12, wherein the pH of the said composition is in the range of 3.0 to 8.0, preferably from 4.5 to 7.5.

14. The Biphasic liquid pharmaceutical composition according to claim 1, wherein the vehicle is selected from the group consisting of purified water, alcohol, polyethylene glycol, propylene glycol or glycerin buffers, oils, acetylated glycerides, ethyl oleate, mineral oil, fatty acid esters, mono or di fatty acid esters of polyethylene glycols, or glyceryl monooleate, alcohol, acetic acid, acetone, ethyl acetates or combinations thereof.

15. The Biphasic liquid pharmaceutical composition according to claim 14, wherein the vehicle is purified water.

16. The Biphasic liquid pharmaceutical composition according to claim 1, wherein a Biphasic liquid pharmaceutical composition is manufactured by mixing and high-shear homogenization method comprising the steps of:
(a) Dissolving preservatives into previously heated purified water, followed by the addition of 30% of the total volume of purified water and the solution was cooled down at room temperature;
(b) Mixing Simethicone emulsion 30% and Carbomer to the solution formed in Step (a) for 10-15 minutes in silverson mixer;
(c) Mixing of an Anticonvulsant drug with glycerol for 10-15 minutes in silverson mixer;
(d) Continuous mixing of the Carbomer-containing solution with the homogenous dispersion of an Anticonvulsant drug of step-(c) for 15-20 minutes in silverson mixer with nitrogen purging;
(e) Homogeneous mixing of Sodium Saccharin with dispersion of Step-(d) for 10-15 minutes in silverson mixer with nitrogen purging;
(f) Measuring the pH of the resulting dispersion of step-(e) and adjusting the pH of the dispersion between 6.7-7.0 with Sodium hydroxide solution for 15-20 minutes in silverson mixer with nitrogen purging;
(g) Mixing of Natural Strawberry flavor with the homogeneous dispersion of Step-(f) for 15-20 minutes in silverson mixer with nitrogen purging;
(h) Adding the volume of final composition with purified water; and
(i) Filling the final dispersion in the amber colored bottle.

17. The Biphasic liquid pharmaceutical composition according to claim 1, wherein the ratio of an Anticonvulsant drug to suspending agent is in the range from about 0.5:1 to about 15:1 and preferably it is in the range from about 1.5:1 to about 10:1.

18. The Biphasic liquid pharmaceutical composition according to claim 1, wherein more than 90% of an Anticonvulsant drug from said composition is released in 15 minutes, preferably more than 90% of an Anticonvulsant drug from said composition is released within 10 minutes.

19. The Biphasic liquid pharmaceutical composition according to claim 1, is used for the treatment of psychiatric conditions which include mainly alcohol and benzodiazepine withdrawal syndromes, panic and anxiety disorders, dementia, schizophrenia, epilepsy, bipolar disorder, partial seizures, primary and secondary tonic-clonic seizures, seizures associated with Lennox-Gastaut syndrome and personality disorders.

20. The Biphasic liquid pharmaceutical composition according to claim 1, wherein an Anticonvulsant drug is selected from the group of consisting Carbamazepine, Oxcarbazepine, Lamotrigine, Phenytoin, Valproicacid, zonisamide, Gabapentin, Levetiracetam, Pregabalin, Clonazepam, Lacosamide, Rufinamide, Vigabatrin, Phenobarbital, Valnoctamide, Perampanel, Pyridoxine or pharmaceutically acceptable salts thereof.

21. The Biphasic liquid pharmaceutical composition according to claim 20, wherein an Anticonvulsant drug is selected from Lamotrigine, phenytoin and Carbamazepine.

22. The Biphasic liquid pharmaceutical composition according to claim 21, wherein the D10 particle size of an Anticonvulsant drug or pharmaceutically acceptable salt thereof, is in the range from about 1.0 μm to 10.0 μm, preferably in the range from about 2.5 μm to 9.0 μm.

23. The Biphasic liquid pharmaceutical composition according to claim 21, wherein the D50 particle size of an Anticonvulsant drug or pharmaceutically acceptable salt thereof, is in the range from about 10 μm to 30 μm, preferably in the range from about 15.0 μm to 25 μm.

24. The Biphasic liquid pharmaceutical composition according to claim 21, wherein the D90 particle size of an Anticonvulsant drug or pharmaceutically acceptable salt thereof, is in the range from about 30 μm to 50 μm, preferably in the range from about 35 μm to 45 μm.
